(19) United States
(12) Patent Application Publication
(10) Pub. No.: US 2023/0334868 A1
(43) Pub. Date: Oct. 19, 2023

(54) SURGICAL PHASE RECOGNITION WITH SUFFICIENT STATISTICAL MODEL

(71) Applicants: THE GENERAL HOSPITAL CORPORATION, Boston, MA (US); MASSACHUSETTS INSTITUTE OF TECHNOLOGY, Cambridge, MA (US)

(72) Inventors: Daniel A. Hashimoto, Cambridge, MA (US); Yutong Ban, Cambridge, MA (US); Thomas M. Ward, Boston, MA (US); Ozanan Meireles, Boston, MA (US); Daniela Rus, Cambridge, MA (US); Guy Rosman, Boston, MA (US)

(21) Appl. No.: 18/023,135

(22) PCT Filed: Aug. 26, 2021
(86) PCT No.: PCT/US2021/047770

§ 371 Date: Feb. 24, 2023

Related U.S. Application Data
(60) Provisional application No. 63/070,698, filed on Aug. 26, 2020.

(51) Int. Cl. G06V 20/52; G16H 40/20; G06N 3/0442; G06V 10/82

(52) U.S. Cl. CPC G06V 20/52; G16H 40/20; G06N 3/0442; G06V 10/82

(57) ABSTRACT

Systems and methods are provided for identifying a current phase of a surgical procedure. Sensor data representing a time period is received and a plurality of numerical features representing the time period are generated from the sensor data. A statistical parameter representing a plurality of stored values from a memory is generated at a sufficient statistics model. An output, representing a surgical phase associated with the time period is provided at a recurrent neural network from a set of inputs that includes the plurality of numerical features and the statistical parameter.

### RELATED APPLICATIONS

The present application claims priority to U.S. Provisional Patent Application Ser. No. 63/070,698 filed Aug. 26, 2020 under Attorney Docket Number MGH 2020-525 and entitled SURGICAL PHASE RECOGNITION WITH A SUFFICIENT STATISTIC MODEL. The entire content of this application is incorporated herein by reference in its entirety for all purposes.

### TECHNICAL FIELD

This disclosure relates to systems and methods for decision support and, in particular, is directed to systems and methods for surgical phase recognition with a sufficient statistic model

### BACKGROUND

As surgical care quality increases with new technologies and greater understanding of surgical disease, gaps remain in both access to and quality of care for many patients. This has led to minimal volume pledges that restrict surgical procedures to surgeons and hospitals with an arbitrarily determined number of sufficient annual cases. Volume pledges have raised concerns over the potential regionalization of surgical care and the impact that regionalization may have on access to surgery, particularly for rural areas. High volume hospitals for complex operations are not readily accessible to many patients, and recent work has shown, for example, that rural patients with cancer are more likely to have their resections performed at a low-volume, yet local, hospital. There is also evidence to suggest that regionalization of care would disproportionately affect minorities and patients without private insurance, as they are most likely to have their operations performed at low-volume hospitals. Thus, the proposed redistribution of care with volume pledges may not be the best solution for all patients.

An estimated 234.2 million operations are performed annually worldwide, but surgeons learn from one patient at a time, limiting their knowledge on rate procedures. Residency is designed to give surgeons the fundamental skills necessary to apply and expand principles of safe surgery to each situation encountered in practice, even novel situations. However, residency relies on apprenticeship-like exposure to experienced surgeons. These experienced surgeons, with a wealth of experiential data, have limited availability. Training for rare cases has thus necessarily been left to a limited number of surgeons who complete sub-specialty fellowships which are often housed in high volume, urban academic centers, again leaving rural and minority populations with a disadvantage in access to care.

Previous attempts have been made to accumulate and distribute intraoperative decision-making models to surgeons to optimize surgical care. Cognitive task analysis (CTA) has been used to codify and distill experienced surgeons' knowledge into standardized checklists to assist in decision-making. In surgical patients, up to 67% of errors occur intraoperatively, and of those errors, 86% of errors are secondary to cognitive factors such as failures in judgment or memory that lead to poor decisions. However, CTA is limited by the fact that 50-75% of decisions made in surgery can be lacking in the conscious recall of surgeons due to either inexperience or automaticity, and these efforts have been time consuming and have not addressed morbidity and mortality at a large scale.

### SUMMARY

In accordance with an aspect of the present invention, a system is provided. A sensor is positioned to monitor a surgical procedure on a patient. The surgical procedure includes a plurality of surgical phases. A non-transitory computer readable medium stores machine executable instructions for determining a current surgical phase. The machine executable instructions are executed by a processor to provide a sensor interface that receives sensor data from the sensor. The sensor data represents a time period of a plurality of time periods comprising the surgical procedure. A feature extractor generates a plurality of numerical features representing the time period from the sensor data. A recurrent neural network receives a set of inputs and provides an output representing a surgical phase associated with the time period of the plurality of surgical phases. The recurrent neural network includes a hidden layer. The set of inputs includes the plurality of numerical features. A memory stores a representation of the hidden layer of the recurrent neural network as one of a plurality of sets of stored values. A sufficient statistics model that generates a statistical parameter representing the plurality of sets of stored values. The statistical parameter is provided as part of the set of inputs.

In accordance with another aspect of the present invention, a method is provided for identifying a current phase of a surgical procedure. Sensor data representing a time period is received and a plurality of numerical features representing the time period are generated from the sensor data. A statistical parameter representing a plurality of stored values from a memory is generated at a sufficient statistics model. An output, representing a surgical phase associated with the time period is provided at a recurrent neural network from a set of inputs that includes the plurality of numerical features and the statistical parameter.

In accordance with yet another aspect of the present invention, a system is provided. A camera is positioned to monitor a surgical procedure on a patient. The surgical procedure includes a plurality of surgical phases. A non-transitory computer readable medium stores machine executable instructions for providing a surgical decision support system. The machine executable instructions are executed by a processor to provide a sensor interface that receives a frame of video from the camera. The frame of video represents a time period of a plurality of time periods comprising the surgical procedure. A convolutional neural network generates a plurality of numerical features representing the time period from the frame of video. A long short term memory (LSTM) network receives a set of inputs and provides an output representing a surgical phase associated with the time period. The LSTM includes a hidden layer. The set of inputs includes the plurality of numerical features. A memory stores a representation of the hidden layer of the recurrent neural network as one of a plurality of sets of stored values. Each of the plurality of sets of stored values represents one of the plurality of time periods. A sufficient statistics model generates a statistical parameter representing the plurality of sets of stored values. The statistical parameter is provided as part of the set of inputs.

### BRIEF DESCRIPTION OF THE DRAWINGS

FIG. 1 illustrates an example of a system for identifying a surgical phase from sensor data;

FIG. 2 is a schematic illustration of one example of a model that could be used with the system of FIG. 1;

FIG. 3 illustrates a method for identifying a current phase of a surgical procedure;

FIG. 4 illustrates a method for identifying a current phase of a surgical procedure; and

FIG. 5 illustrates a computer system that can be employed to implement systems and methods described herein.

### DETAILED DESCRIPTION

The systems and methods presented herein seek to boost the effective experience of surgeons by data mining operative sensor data, such as video, to generate a collective surgical experience that can be utilized to provide automated predictive-assistive tools for surgery. Rapid advancements in streaming data analysis have opened the door to efficiently gather, analyze, and distribute collective surgical knowledge. However, simply collecting massive amounts of data is insufficient, and human analysis at the individual case level is costly and time-consuming. Therefore, any real solution must automatically summarize many examples to reason about rare, yet consequential, events that occur in surgery.

The future of computer-assisted laparoscopic surgery relies upon strong fundamental automated understanding of surgical workflow from videos. While significant work has been performed in improving the understanding of video and producing better annotation and supervision cues, existing models still fall short of a complete and automatic interpretation of surgery. Unlike the progress made in interpreting images from reconstructive modalities such as Computed Tomography (CT) or Magnetic Resonance Imaging (MRI), surgery is a temporal process with only weakly observable visual-cues which requires reasoning over the whole temporal process.

Understanding surgical workflows requires reasoning about events across highly varied temporal scales, from a few seconds to few hours, which exceeds the capabilities of existing models. As a brief example, in laparoscopic cholecystectomy, “Dissection of Calot's triangle” involves removing the lower portion of the gallbladder from the liver bed (i.e. clearing the cystic plate). This phase can be visually indistinct from “Removal of the Gallbladder from the Liver Bed” later in the case and requires knowledge that key phases (which happen minutes later) have not yet been performed to accurately infer the current surgical phase. In such cases, information extracted by LSTM remains local compared to the total duration of the surgery and fails to improve classification performance. Accordingly, the systems and methods presented herein utilize a sufficient statistics model to retain information about the progression of the surgery allowing for more effective identification of the current surgical phase of a surgical procedure.

As used herein, a “surgical phase” or “surgical phase” is a period of time within a surgical procedure in which an action or set of related actions is taken by the surgeon. In general, surgical phases are sequential, although it will be appreciated that the order of some the surgical phases can vary for a given procedure and that some phases can be interrupted by another phase, such that they appear more than once in a given sequence. Two examples of surgical phases that can be considered during a cholecystectomy are listed in Tables 1 and 2.

As used herein, a statistic is “sufficient” with respect to a statistical model and its associated unknown parameter if no other statistic that can be calculated from the same sample provides any additional information as to the value of the parameter. In particular, a statistic is sufficient for a family of probability distributions if the sample from which it is calculated gives no additional information than the statistic, as to which of those probability distributions is the sampling distribution. A “sufficient statistics model” is a statistical model that can generate one or more approximations of sufficient statistics from a data set that represent an unknown parameter of the dataset.

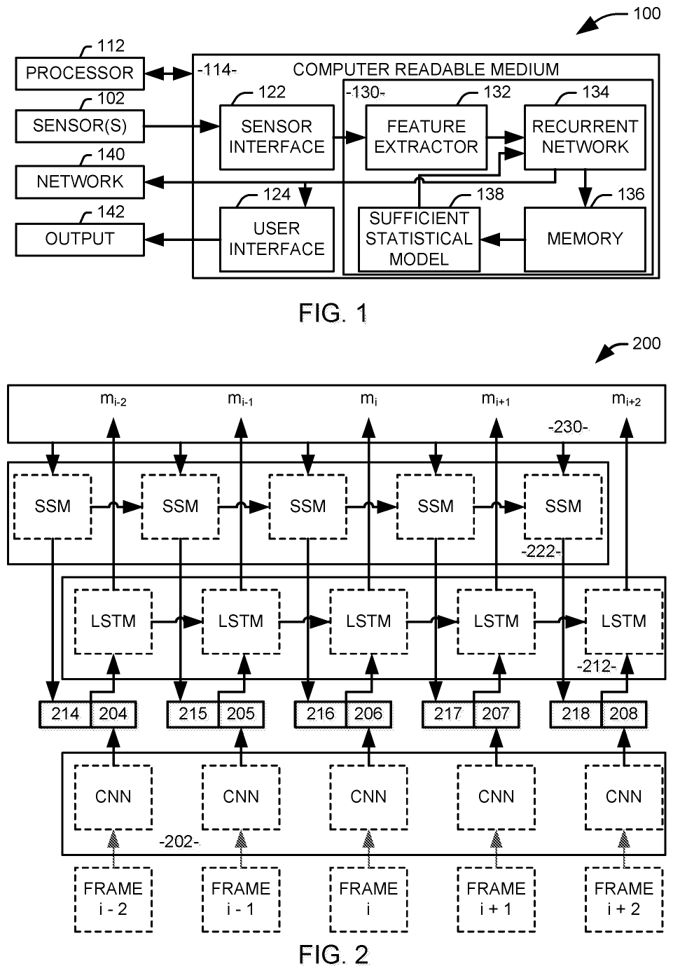

FIG. 1 illustrates an example of a system 100 for identifying a surgical phase from sensor data. The system 100 includes at least one sensor 102 positioned to monitor a surgical procedure on a patient. Sensors, for this purpose, can include video cameras, in the visible or infrared range, a microphone or other input device to receive comments from the surgical team at various time points within the surgery, accelerometers or radio frequency identification (RFID) devices disposed on a surgeon or an instrument associated with the surgical procedure, intraoperative imaging technologies, such as optical coherence tomography, computed tomography, X-ray imaging, sensor readings from other systems utilized in the surgical procedure, such as an anesthesia system, and sensors that detect biometric parameters of the patient, such as sphygmomanometers, in vivo pressure sensors, pulse oximeters, and electrocardiographs.

A non-transitory computer readable medium 112 stores machine executable instructions that are executed by an associated processor 114. It will be appreciated, however, that the system 100 could instead be implemented as dedicated hardware or programmable logic, or that the non-transitory computer readable medium 112 could comprise multiple, operatively connected, non-transitory computer readable media that are each either connected locally to the processor 114 or connected via a network connection.

The executable instructions stored on the non-transitory computer readable medium 112 include a sensor interface 122 that receives and conditions data from the at least one sensor 102, a user interface 124, and a model 130. The model 130 represents the surgical procedure as a progression through a set of states, referred to herein as “surgical states” or “surgical phases.” The set of surgical states can either be selected in advance, for example, by a human expert or learned as a non-parametric inference during training of the model 130.

The model 130 includes a feature extractor 132 that receives sensor data from the at least one sensor 102 representing a specific time period, i. In one implementation, the sensor data for a given time period is a frame of video captured during the surgery. The feature extractor 132 reduces the sensor data into an output vector comprising a plurality of values representing the content of the sensor data. In particular, the feature extractor 132 extracts a plurality of features, which can be categorical, discrete, and continuous parameters representing the sensor data. In one example, the parameters can include descriptive statistics, such as measures of central tendency (e.g., median, mode, arithmetic mean, or geometric mean) and measures of deviation (e.g., range, interquartile range, variance, standard deviation, etc.) of time series of various parameters represented in the sensor data.

In one example, the feature extractor 132 is a convolutional neural network that includes convolutional layers in which nodes from a previous layer of the network are only connected to a subset of the nodes in the convolutional layer. These convolutional layers can be used to extract features from sensor data, such as audio and images. In particular, the convolutional neural network can be trained on data labelled with an appropriate output class, in this case, a surgical state represented by the sensor data, to learn useful features for extraction, such that the output vector provided by convolutional neural network is a reduced dimensionality representation of the sensor data.

The output of the feature extractor 132 is provided to a recurrent neural network 134. Recurrent neural networks are a class of neural networks in which connections between nodes form a directed graph along a temporal sequence. Unlike a feedforward network, recurrent neural networks can incorporate feedback from states caused by earlier inputs, such that an output of the recurrent neural network for a given input can be a function of not only the input but one or more previous inputs. For example, Long Short-Term Memory (LSTM) networks are a modified version of recurrent neural networks, which makes it easier to selectively remember past data in memory.

The recurrent neural network 134 provides an output representing the current surgical phase, that is, the surgical phase associated with the specific time, i. In one implementation, the output of the recurrent neural network 134 is a vector of values, each representing a likelihood that one of the set of surgical states is the current surgical state. For example, the recurrent neural network 134 can include a final dense layer using a softmax activation to generate these values. It will be appreciated that, as a recurrent neural network, at least some hidden values from each iteration of the recurrent neural network 134 are retained for the next iteration, such that the output associated with the i^{th} time period depends at least in part on the input from the (i−1)^{th} time period. Further, in each iteration, at least one value associated with a hidden layer of the recurrent neural network 134 can be stored in a memory 136 to represent a state of the recurrent neural network during that time period. In one implementation, a transform is applied to the values in the hidden layer to provide the set of values to be stored in the memory 136. It will be appreciated that while recurrent neural networks, such as long short term memory networks, have some degree of internal memory that allows the state of the network, the memory 136 is independent of this internal memory and can store different information than the hidden states retained between iterations of the recurrent neural network. In one example, the transform is provided by the dense layer of the recurrent neural network 134, such that the output of the recurrent neural network is stored in the memory 136 as the set of values.

A sufficient statistic model 138 that summarizes the sets of values stored in the memory 136 as a set of statistics. The set of statistics can be provided as an additional input to the recurrent neural network 134 in determining the current surgical state. In one implementation, the current surgical state is determined in real-time, and the sufficient statistic model 138 updates the set of statistics as each set of sensor data is evaluated. Accordingly, the set of statistics can be determined from the values stored in the memory 136 at the current time period, which each represent a time period preceding the current time period. Alternatively, the set of statistics can be determined after a surgery, in which case the set of statistics used as an input with the sensor data associated with a given time period can be determined from stored values representing time periods both preceding and following the time period.

In one implementation, the sufficient statistic model 138 includes a hidden Markov model. A hidden Markov model models observed data as a series of outputs generated by one of several hidden internal states. Along with the observations, a hidden Markov model can include rules for transitioning among states, such that information beyond the observations can be employed to identify state transitions. Using the values extracted from the hidden layer of the recurrent neural network 134 as observations, one or more values representing the probability that the surgery is in a given surgical state can be determined from the hidden Markov model and included as all or part of the set of statistics provided as an input to the hidden Markov model.

Additionally or alternatively, the sufficient statistics model 138 can determine a cumulative sum likelihood of probability values extracted from the hidden layer of the recurrent neural network 134. It will be appreciated that the output of the recurrent neural network 134 can either be formatted as a likelihood or easily transformed into likelihood values, and in one example, the output of the recurrent neural network is used to compute the cumulative sum likelihood. The cumulative sum likelihood at time i, tᵢ can be computed as:

fᵢ=log[Σₜ₌₁ⁱ=( [symbol] /(mₜ))+1] Eq. 1

where ( [symbol] (mₜ)) represents a thresholding of the elements of the values, mₜ, derived from the hidden layer using a set of threshold levels, I, with respect to the surgical phase having a maximum probability at time i.

The cumulative sum likelihood feature enhances understanding of global context and allows the network 134 to capture both maximum-probability and probable interpretation of the surgical state at a given time. It can indicate if certain phases have or have not already occurred. For example, in a cholecystectomy procedure, the conditional sum likelihood can indicate if a division of the cystic duct has already been achieved. Since it is a non-repeated event, future frames cannot be classified as such thereafter.

Additionally or alternatively, the sufficient statistics model 138 can apply one or more wavelet transform to summarize temporal information from the data stored in the memory 136. In one implementation, a filter bank with Gabor filters of different Gaussian envelope sizes can be directly applied to the likelihood space along the time axis. In one example of this implementation, ten different filters having kernel sizes ranging from ten to thirty were applied. The filtered results are then concatenated together to gather the temporal information of different time scales. It will be appreciate that other wavelet transforms, as well as Gabor filters of different kernel sizes, can be used. For example, while the Gabor representation is O(T) compute as described, there are efficient approximations for both Gabor and other wavelets. For example, Haar wavelets are trivial to compute at O(1) complexity using integral images.

The determined surgical phase can be provided to a network interface 140 that communicates the determined surgical phase to a surgical decision support system (not shown). One example of a surgical decision support system with which the system 100 can be employed can be found in U.S. Published Patent Application No. 2020/0170710, entitled “SURGICAL DECISION SUPPORT USING A DECISION THEORETIC MODEL,” the entire contents of which are hereby incorporated by reference. In one implementation, the selected surgical states can be associated with corresponding resources, and the network interface 140 can notify appropriate personnel in advance that various resources are expected to be required or desired. For example, if the recurrent neural network 134 determines that a surgical state for which radiological imaging is desirable imminently follows the current surgical state, the network interface 140 could transmit a message to a member of the operating team or another individual at the facility in which the surgical procedure is performed to request the necessary equipment. Similarly, if the recurrent neural network 134 predicts a progression through the surgical states that diverges from an expected progression, the network interface 140 could transmit a message to a coordinator for the facility in which the surgical procedure is performed to schedule additional time in the operating room. Accordingly, the system 100 can be used to more efficiently allocate resources across a surgical facility.

Additionally or alternatively, the surgical state indicated by recurrent neural network 134 can be provided to a human being via an appropriate user interface 124 and output device 142, such as a video monitor, speaker, or network interface. It will be appreciated that the current surgical phase, and predictions derived therefrom by the surgical assisted decision making system, can be provided directly to the surgeon to guide surgical decision making. For example, if a complication or other negative outcome is anticipated without additional radiological imaging, the surgeon could be advised to wait until the appropriate imaging can be obtained. Thus, the system 100 can be employed to assist less experienced surgeons in less common surgical procedures or unusual presentations of more common surgical procedures.

FIG. 2 is a schematic illustration of one example of a model 200 that could be used with the system of FIG. 1. The model 200 represents the analysis of five time periods of sensor input (tᵢ₋₂ to t₁₊₂) and illustrates the interrelationship of the various components of the model over this time. Each input is provided to the convolutional neural network 202 to produce respective sets of data 204-208 representing the visual content. A sufficient statistics model (SSM) 212 provides a set of statistics 214-218 that are concatenated onto the visual content data 204-208 to provide an input vector for the long short term memory (LSTM) network 222. The LSTM network 222, at each iteration, generates an output representing the current surgical state and passes one or more hidden states to a next iteration of the LSTM network.

The model 200 also takes the past hidden LSTM layer and passes it through a transform to get a vector mₜ, conceptualized as a temporal vector signal Mₜ={m1 . . . mₜ} in a memory 230. It then computes aggregate statistics of the transformed signal at the sufficient statistics model 212, resulting in a sufficient statistics feature stream S={s₁ . . . sₜ}, such that the sufficient statistics model is updated with new values at each iteration. By concatenating these statistics with the output, the model 200 then feeds them to the current time LSTM network iteration as an augmented feature vector. After concatenation, the LSTM network 222 is applied taking the augmented feature vector as an input to output the likelihood for each phase. For both training and testing, the memory is initialized with a pretrained CNN+LSTM model. After the first iteration, the new LSTM network prediction updates the memory 230.

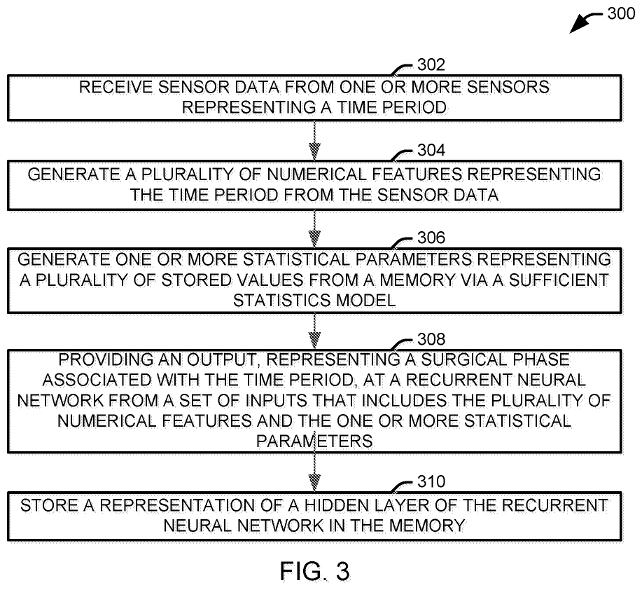

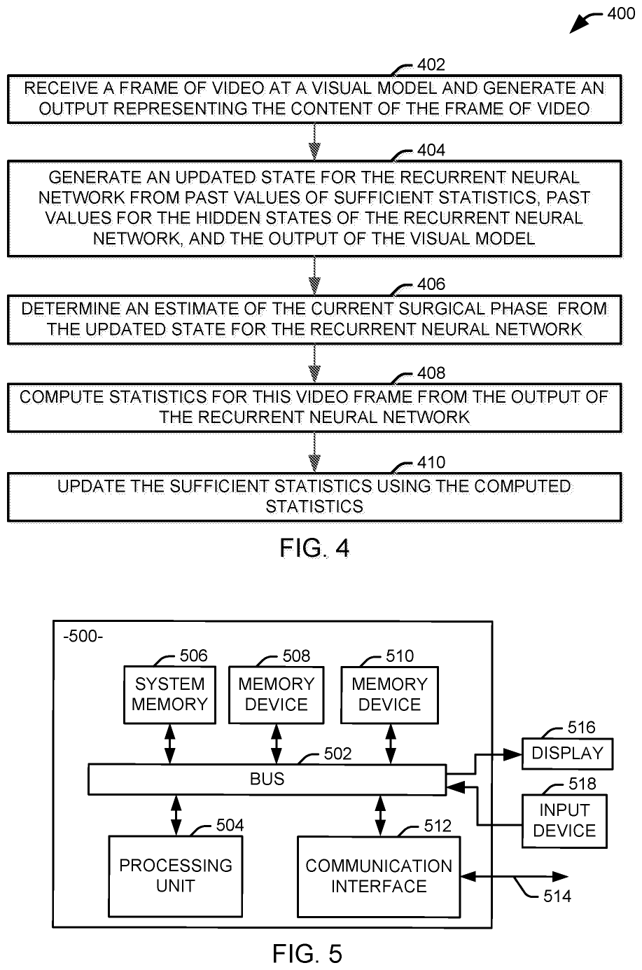

In view of the foregoing structural and functional features described above, methods in accordance with various aspects of the invention will be better appreciated with reference to FIGS. 3 and 4. While, for purposes of simplicity of explanation, the methods of FIGS. 3 and 4 are shown and described as executing serially, it is to be understood and appreciated that the invention is not limited by the illustrated order, as some aspects could, in accordance with the invention, occur in different orders and/or concurrently with other aspects from that shown and described herein. Moreover, not all illustrated features may be required to implement a method in accordance with an aspect of the invention. The example methods of FIGS. 3 and 4 can be implemented as machine-readable instructions that can be stored in a non-transitory computer readable medium, such as can be computer program product or other form of memory storage. The computer readable instructions corresponding to the methods of FIGS. 3 and 4 can also be accessed from memory and be executed by a processing resource (e.g., one or more processor cores).

FIG. 3 illustrates a method 300 for identifying a current phase of a surgical procedure. It will be appreciated that the method will be implemented by an electronic system, which can include any of dedicated hardware, machine executable instructions stored on a non-transitory computer readable medium and executed by an associated processor, or a combination of these. In practice, the model used by the method will have already been trained on sensor data from a set of previously performed surgical procedures via a supervised or semi-supervised learning process. At 302, sensor data is received from one or more sensors representing a time period of a plurality of time periods comprising a surgical procedure. In one implementation, the sensor data is a frame of video captured at a camera.

At 304, a plurality of numerical features representing the time period are generated from the sensor data. In one implementation, the sensor data is provided to a convolutional neural network to provide the plurality of numerical features. At 306, one or more statistical parameters representing a plurality of stored values from a memory is generated via a sufficient statistics model. Each of the plurality of stored values representing a time period preceding the time period. In one example, the sufficient statistics model applies a wavelet decomposition to the set of stored values to provide a set of wavelet coefficients. In another example, the sufficient statistics model comprises a hidden Markov model that receives the set of stored values as observations. In a third example, the sufficient statistics model generates a cumulative sum likelihood from the set of stored values. It will be appreciated that these examples are not mutually exclusive, and that multiple methods can be applied to provide the one or more statistical parameters.

At 308, an output, representing a surgical phase associated with the time period of the plurality of surgical phases, is provided at a recurrent neural network from a set of inputs that includes the plurality of numerical features and the one or more statistical parameters. In one example, the recurrent neural network is implemented as a long short term memory network. The resulting output can be displayed to a user or provided to a surgical assisted decision making system. In one example, a message can be transmitted to an individual at the facility in which the surgical procedure is performed via a network interface to request an item of equipment in response to the output. At 310, a representation of a hidden layer of the recurrent neural network is stored in the memory as one of the plurality stored values. This representation can be an output of the recurrent neural network in the memory or a set of transformed values generated by applying a transform to a set of values stored in the hidden layer.

FIG. 4 illustrates another method 400 for identifying a current phase of a surgical procedure. In particular, the method 400 receives a set of past hidden states, for a recurrent neural network, a past value for one or more sufficient statistics representing data from the recurrent neural network stored in a memory, and a frame of video and provides an updated value for the sufficient statistics, a new hidden state value or values, and an estimate of the current phase of the surgical procedure. At 402, a visual model receives the frame of video and generates an output representing the content of the frame of video. In one example, where the sensor is a video camera, the observations are generated via a visual model, implemented as a discriminative classifier model that interprets the visual data. This interpretation can be indirect, for example, by finding objects within the scene that are associated with specific surgical states or world states, or by directly determining a surgical state or world state via the classification process. In one example, the visual model is implemented as an artificial neural network, such as a convolutional neural network, a cluster network, or a recurrent neural network, that is trained on the plurality of time series of observations to identify the surgical state. Since the system is intended to learn from a limited amount of data and under small computational resource, a feature space for generating observations is selected to be concise and representative, with a balance between invariance and expressiveness.

In another implementation, the classification is performed from several visual cues in the videos, categorized broadly as local and global descriptor and motivated by the way surgeons deduce the phase of the surgery. These cues are used to define a feature space that captures the principal axes of variability and other discriminant factors that determine the surgical state, and then the discriminative classifier can be trained on a set of features comprising the defined feature space. The cues include color-oriented visual cues generated from a training image database of positive and negative images. Other descriptor categories for individual RGB/HSV channels can be utilized to increase dimensionality to discern features that depend on color in combination with some other property. Pixel values can also be used as features directly. The RGB/HSV components can augment both local descriptors (e.g., color values) and global descriptors (e.g., a color histogram). The relative position of organs and instruments is also an important visual cue. The position of keypoints generated via speeded-up robust features (SURF) process can be encoded with an 8×8 grid sampling of a Gaussian surface centered around the keypoint. The variance of the Gaussian defines the spatial “area of influence” of a keypoint.

Shape is important for detecting instruments, which can be used as visual cues for identifying the surgical state, although differing instrument preferences among surgeons can limit the value of shape-based cues. Shape can be encoded with various techniques, such as the Viola-Jones object detection framework, using image segmentation to isolate the instruments and match against artificial 3D models, and other methods. For local frame descriptors, a standard SURF descriptor can be used as a base, and for a global frame descriptor, grid-sampled histogram of ordered gradients (HOG) descriptors and discrete cosign transform (DCT) coefficients can be added. Texture is a visual cue used to distinguish vital organs, which tend to exhibit a narrow variety of color. Texture can be extracted using a co-occurrence matrix with Haralick descriptors, by a sampling of representative patches to be evaluated with a visual descriptor vector for each patch, and other methods. In one example, a Segmentation-based Fractal Texture Analysis (SFTA) texture descriptor is used.

At 404, an updated state for the recurrent neural network is generated from the past values of the sufficient statistics, the past values for the hidden states of the recurrent neural network, and the output of the visual model. At 406, an estimate of the current surgical phase is determined from the updated state for the recurrent neural network. For example, where the output of the recurrent neural network can be provided to a dense layer to provide a value representing the current surgical phase. At 408, statistics for the current video frame are computed from the output of the recurrent neural network, and at 410, these frame statistics are used to update the sufficient statistics for use in determining a next surgical phase.

FIG. 5 is a schematic block diagram illustrating an exemplary system 500 of hardware components capable of implementing examples of the systems and methods disclosed herein. The system 500 can include various systems and subsystems. The system 500 can be a personal computer, a laptop computer, a workstation, a computer system, an appliance, an application-specific integrated circuit (ASIC), a server, a server BladeCenter, a server farm, etc.

The system 500 can include a system bus 502, a processing unit 504, a system memory 506, memory devices 508 and 510, a communication interface 512 (e.g., a network interface), a communication link 514, a display 516 (e.g., a video screen), and an input device 518 (e.g., a keyboard, touch screen, and/or a mouse). The system bus 502 can be in communication with the processing unit 504 and the system memory 506. The additional memory devices 508 and 510, such as a hard disk drive, server, standalone database, or other non-volatile memory, can also be in communication with the system bus 502. The system bus 502 interconnects the processing unit 504, the memory devices 506-510, the communication interface 512, the display 516, and the input device 518. In some examples, the system bus 502 also interconnects an additional port (not shown), such as a universal serial bus (USB) port.

The processing unit 504 can be a computing device and can include an application-specific integrated circuit (ASIC). The processing unit 504 executes a set of instructions to implement the operations of examples disclosed herein. The processing unit can include a processing core. The additional memory devices 506, 508, and 510 can store data, programs, instructions, database queries in text or compiled form, and any other information that may be needed to operate a computer. The memories 506, 508 and 510 can be implemented as computer-readable media (integrated or removable), such as a memory card, disk drive, compact disk (CD), or server accessible over a network. In certain examples, the memories 506, 508 and 510 can comprise text, images, video, and/or audio, portions of which can be available in formats comprehensible to human beings. Additionally or alternatively, the system 500 can access an external data source or query source through the communication interface 512, which can communicate with the system bus 502 and the communication link 514.

In operation, the system 500 can be used to implement one or more parts of a system in accordance with the present invention. Computer executable logic for implementing the diagnostic system resides on one or more of the system memory 506, and the memory devices 508 and 510 in accordance with certain examples. The processing unit 504 executes one or more computer executable instructions originating from the system memory 506 and the memory devices 508 and 510. The term “computer readable medium” as used herein refers to a medium that participates in providing instructions to the processing unit 504 for execution. This medium may be distributed across multiple discrete assemblies all operatively connected to a common processor or set of related processors.

Specific details are given in the above description to provide a thorough understanding of the embodiments. However, it is understood that the embodiments can be practiced without these specific details. For example, physical components can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps, and means described above can be done in various ways. For example, these techniques, blocks, steps, and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine-readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” can represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes but is not limited to portable or fixed storage devices, optical storage devices, wireless channels, and/or various other storage mediums capable of storing that contain or carry instruction(s) and/or data.

In the preceding description, specific details have been set forth in order to provide a thorough understanding of example implementations of the invention described in the disclosure. However, it will be apparent that various implementations may be practiced without these specific details. For example, circuits, systems, networks, processes, and other components may be shown as components in block diagram form in order not to obscure the example implementations in unnecessary detail. In other instances, well-known circuits, processes, algorithms, structures, and techniques may be shown without unnecessary detail in order to avoid obscuring the examples. The description of the example implementations will provide those skilled in the art with an enabling description for implementing an example of the invention, but it should be understood that various changes may be made in the function and arrangement of elements without departing from the spirit and scope of the invention. Accordingly, the present invention is intended to embrace all such alterations, modifications, and variations that fall within the scope of the appended claims.

What is claimed is:

1. A system comprising:
a sensor positioned to monitor a surgical procedure on a patient, the surgical procedure comprising a plurality of surgical phases;
a processor; and
a non-transitory computer readable medium stores machine executable instructions for providing a surgical decision support system, the machine executable instructions being executed by the processor to provide:
a sensor interface that receives sensor data from the sensor, the sensor data representing a time period of a plurality of time periods comprising the surgical procedure;
a feature extractor that generates a plurality of numerical features representing the time period from the sensor data;
a recurrent neural network, comprising a hidden layer, that receives a set of inputs and provides an output representing a surgical phase associated with the time period of the plurality of surgical phases, the set of inputs including the plurality of numerical features;
a memory that stores a representation of the hidden layer of the recurrent neural network as one of a plurality of sets of stored values; and
a sufficient statistics model that generates a statistical parameter representing the plurality of sets of stored values, the statistical parameter being provided as part of the set of inputs.

2. The system of claim 1, wherein the at least one sensor comprises a camera that captures frame of video.

3. The system of claim 1, wherein the feature extractor comprises a convolutional neural network.

4. The system of claim 1, further comprising a network interface that provides the output representing the surgical phase associated with the time period to a surgical assisted decision making system.

5. The system of claim 1, wherein the recurrent neural network is a long short term memory network.

6. The system of claim 1, wherein the sufficient statistics model applies a wavelet decomposition to the plurality of sets of stored values to provide a set of wavelet coefficients, the set of inputs comprising a wavelet coefficient of the set of wavelet coefficients.

7. The system of claim 1, wherein the sufficient statistics model comprises a hidden Markov model that receives the plurality of sets of stored values as observations, the set of inputs comprising a probability value associated with the hidden Markov model.

8. The system of claim 1, wherein the sufficient statistics model generates a cumulative sum likelihood from the plurality of sets of stored values.

9. The system of claim 1, wherein the set of stored values represents only a set of time periods of the plurality of time periods that precede the time period.

10. The system of claim 1, wherein the set of stored values represents all of the plurality of time periods.

11. A method comprising:
receiving sensor data from the sensor, the sensor data representing a time period of a plurality of time periods comprising a surgical procedure;
generating a plurality of numerical features representing the time period from the sensor data;
generating a statistical parameter representing a plurality of stored values from a memory at a sufficient statistics model;
providing an output, representing a surgical phase associated with the time period of the plurality of surgical phases, at a recurrent neural network from a set of inputs that includes the plurality of numerical features and the statistical parameter.

12. The method of claim 11, further comprising storing a representation of a hidden layer of the recurrent neural network in the memory as one of the plurality stored values.

13. The method of claim 12, wherein storing a representation of the hidden layer of the recurrent neural network comprises storing an output of the recurrent neural network in the memory.

14. The method of claim 12, wherein storing a representation of the hidden layer of the recurrent neural network comprises:
applying a transform to a set of values stored in the hidden layer to provide a set of transformed values; and
storing the set of transformed values in the memory.

15. The method of claim 11, further comprising transmitting, at a network interface, a message to an individual at the facility in which the surgical procedure is performed to request an item of equipment in response to the output.

16. A system comprising:
a camera positioned to monitor a surgical procedure on a patient, the surgical procedure comprising a plurality of surgical phases;
a processor; and
a non-transitory computer readable medium stores machine executable instructions for providing a surgical decision support system, the machine executable instructions being executed by the processor to provide:
a sensor interface that receives a frame of video from the camera, the frame of video representing a time period of a plurality of time periods comprising the surgical procedure;
a convolutional neural network that generates a plurality of numerical features representing the time period from the frame of video;
a long short term memory (LSTM) network, comprising a hidden layer, that receives a set of inputs and provides an output representing a surgical phase of the plurality of surgical phases associated with the time period, the set of inputs including the plurality of numerical features;
a memory that stores a representation of the hidden layer of the recurrent neural network as one of a plurality of sets of stored values, each of the plurality of sets of stored values representing one of the plurality of time periods; and
a sufficient statistics model that generates a statistical parameter representing the plurality of sets of stored values, the statistical parameter being provided as part of the set of inputs.

17. The system of claim 16, wherein the sufficient statistics model generates a cumulative sum likelihood from the plurality of sets of stored values and applies a wavelet decomposition to the plurality of sets of stored values to provide a set of wavelet coefficients, the set of inputs comprising a wavelet coefficient of the set of wavelet coefficients and a value derived from the cumulative sum likelihood.

18. The system of claim 17, wherein the sufficient statistics model further comprises a hidden Markov model that receives the plurality of sets of stored values as observations, the set of inputs further comprising a probability value associated with the hidden Markov model.

19. The system of claim 16, further comprising a user interface that provides the output representing the surgical phase to a human operator.

20. The system of claim 16, further comprising a network interface that provides, in response to the output representing the surgical phase, a message to an individual at the facility in which the surgical procedure is performed to request an item of equipment.
